(19) United States
(12) Plant Patent Application Publication
(10) Pub. No.: US 2019/0246543 P1
(43) Pub. Date: Aug. 8, 2019

(54) Crapemyrtle plant named 'DJ 06-14'

(50) ;

(71) Applicant: University of Georgia Research Foundation, Inc., Athens, GA (US)

(72) Inventors: Matthew A. Dirr, Watkinsville, GA (US); Donglin Zhang, Watkinsville, GA (US)

(73) Assignee: University of Georgia Research Foundation, Inc., Athens, GA (US)

(21) Appl. No.: 16/350,967

(22) Filed: Feb. 5, 2019

Related U.S. Application Data
(60) Provisional application No. 62/627,070, filed on Feb. 6, 2018.

(51) Int. Cl. A01H 6/00

(52) U.S. Cl. PLT/252

(57) ABSTRACT

A new crapemyrtle plant has deep, uniform purple foliage and stems; a deep raspberry pink/red flower color; an intermediate upright growth habit; and powdery mildew and Cercospera resistance.

### CROSS REFERENCE TO RELATED APPLICATION

This application claims the benefit of U.S. Provisional Application Ser. No. 62/627,070, entitled CRAPEMYRTLE PLANT NAMED ‘DJ 06-14’, filed on Feb. 6, 2018.

Latin name of the genus and species of the plant claimed: ‘DJ 06-14’ is a Crapemyrtle plant that is a Lagerstroemia hybrid.

Variety denomination: The new crapemyrtle plant claimed is a variety denominated ‘DJ 06-14’.

### BACKGROUND OF THE INVENTION

The present invention relates to the discovery of a new and distinct cultivar of the ornamental flowering shrub Lagerstroemia indica x Lagerstroemia fauriei, commonly known as crapemyrtle, and hereafter referred to by the varietal denomination ‘DJ 06-14’, as herein described and illustrated.

The new crapemyrtle originated from open pollinated seed of a patented plant, ‘Chocolate Mocha’ Crapemyrtle (U.S. Plant Pat. No. 21,450). ‘DJ 06-14’ was selected from plants grown at the University of Georgia Horticulture Farm in Watkinsville, Ga. The seedlings were planted in containers and selections were made for plants based on the following criteria:

1.) Intermediate, upright growth habit
2.) Powdery mildew resistance
3.) Flower color and quality
4.) Deep purple foliage and stem color
‘DJ 06-14’ was selected in September 2014.

Asexual reproduction by traditional vegetative cuttings since 2014 at the Horticulture Farm in Watkinsville, Ga. has shown that the distinguishing characteristics of the new crapemyrtle variety, ‘DJ 06-14’ are stable and reproduced true to type in successive generations.

### SUMMARY OF THE INVENTION

‘DJ 06-14’ has not been observed under all possible environmental conditions. The phenotype may vary somewhat with changes in light, temperature, soil and rainfall amount, however, any variance in genotype.

The following traits have been observed and represent the characteristics of the new cultivar. Asexual propagation by cuttings has proven that these characteristics are firmly fixed in succeeding asexually propagated generations. In combination, these characteristics distinguish ‘DJ 06-14’ from all other varieties known to the inventor:

1.) Deep, uniform, purple foliage and stems;
2.) Deep raspberry pink/red flower color;
3.) Intermediate, upright growth habit; and
4.) Powdery mildew and Cercospera resistance.

‘DJ 06-14’ is distinguished from its parent plant Lagerstroemia indica x Lagerstroemia fauriei ‘Chocolate Mocha’ by its improved, more consistent, deeper purple foliage and its larger, raspberry pink/red flower color. ‘DJ 06-14’ over 3 years of evaluation, has proven to be more resistant to powdery mildew and Cercospera than ‘Chocolate Mocha’.

### DESCRIPTION OF THE DRAWINGS

The accompanying colored photographic illustrations show the overall appearance and distinct characteristics of the new ‘DJ 06-14’ cultivar. The colors in the photographs are as close as possible with the photographic and printing technology utilized.

FIG. 1 is a close up view of the flowers of the ‘DJ 06-14’ variety.

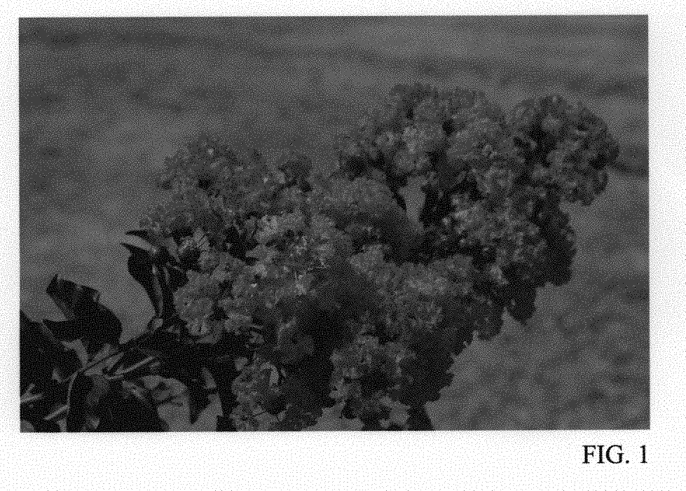

FIG. 2 is a view of a flowering ‘DJ 06-14’ plant.

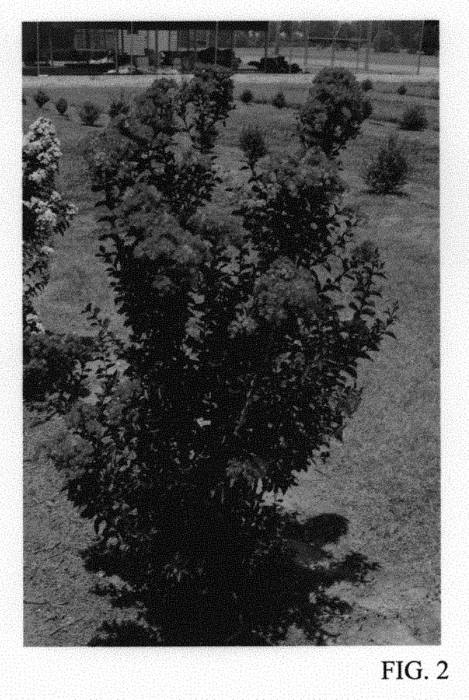

### DETAILED DESCRIPTION

In the following description, color references are made to The Royal Horticulture Society Colour Chart, 2015 Edition, except where general terms of ordinary dictionary significance are used. Plants used for the description were approximately 2.5 years old and grown in 11.4 liter containers in full sun at the UGA Horticulture Farm in Watkinsville, Ga.
Botanical/plant description: ‘DJ 06-14’ is a cultivar of Lagerstroemia indica x Lagerstroemia fauriei ‘Chocolate Mocha’. The current variety is a progeny from seed collected from ‘Chocolate Mocha’ in Nov. of 2013. Male, or pollen parent is unknown (open-pollinated).
Propagation: Vegetatively by terminal cuttings. Time to initiate roots during summer takes about 21 days at 32 degrees C. Flowering shrub; compact, upright, intermediate growth habit. Freely branching; lateral branches abundant without pruning (although some winter pruning of dead wood is recommended). Ultimately, proper pruning will result in a fine-textured, dense habit.
Root description: Numerous, fine, fibrous and well branched.
Plant size: The original plant has been in the ground for 3.5 years, is 152 cm high from the soil level to the top of the uppermost inflorescences and about 91 cm wide. Typical first year stems have a length of from 32 cm to 47 cm and a cross-sectional dimension of typically about 2.5 mm. in cross-section, the first year stems tend to be somewhat square. Typical second year stems and older stems have a diameter of about 4 mm or more. Typical second year stems have a length of 63 cm to 92 cm.

We claim:

1. A new and distinct cultivar of the Crapemyrtle plant named ‘DJ 06-14’, as herein illustrated and described.
